(19) United States
(12) Patent Application Publication
(10) Pub. No.: US 2020/0327991 A1
(43) Pub. Date: Oct. 15, 2020

(54) SYSTEMS AND METHODS FOR DETERMINING RELAPSE RISK AND PROVIDING REWARDS

(71) Applicant: Pala-linq Social Purpose Corporation, Seattle, WA (US)

(72) Inventors: Paul Campbell, Seattle, WA (US); Will Wright, Seattle, WA (US); Daniela Luzi Tudor, Seattle, WA (US)

(73) Assignee: Pala-linq Social Purpose Corporation, Seattle, WA (US)

(21) Appl. No.: 16/843,134

(22) Filed: Apr. 8, 2020

Related U.S. Application Data
(60) Provisional application No. 62/831,558, filed on Apr. 9, 2019.

(51) Int. Cl. G16H 50/30; G06Q 30/02

(52) U.S. Cl. CPC G16H 50/30; G06Q 30/0208; G06Q 30/0225

(57) ABSTRACT

Disclosed herein are systems and methods for determining an individual in addiction recovery's risk of relapse in the form of a risk score. The risk score may be a quantitative assessment of the individual's risk of relapse. The risk score may be based on a variety of inputs including GPS-verified activity check ins. The inputs may be used to calculate a set of risk factors. The risk factors may then be weighted and summed to generate the risk score. The systems and methods may further generate an adjusted risk score that sets sensitivities for the risk factors. The systems and methods may rank individuals based on their risk scores or adjusted risk scores. The risks scores and adjusted risk scores may be provided to a case manager responsible for individuals' care. Systems and methods for providing rewards to individuals based on compliance are also disclosed.

### CROSS-REFERENCE TO RELATED APPLICATION(S)

This application claims the benefit under 35 U.S.C. § 119 of the earlier filing date of U.S. Provisional Application Ser. No. 62/831,558 filed Apr. 9, 2019, the entire contents of which are hereby incorporated by reference in their entirety for any purpose.

### BACKGROUND

According to the National Survey on Drug Use and Health provided by the National institute on Drug Abuse by the National Institute of Health, over 20 million Americans struggled with addiction in 2014. While only a fraction of those needing treatment actually receive it, recovery treatment may be labor intensive. Individuals in recovery may need to engage in many support activities such as appointments with medical professionals and group meetings (e.g., Alcoholics Anonymous). Case managers may coordinate care for several individuals including scheduling support activities, meeting with individuals personally, and checking in remotely (e.g., via phone or email).

Case managers may have to rely on qualitative factors and intuition to determine which of the individuals under their care need more attention. As the case manager's caseload increases, the ability of the case manager to prioritize the needs of the individuals may become compromised and individuals at the highest risk for relapse may not receive the attention they need to stay in recovery. Accordingly, improved techniques to assist case managers prioritize their caseload is desired.

Furthermore, research has shown that positive reinforcement by providing rewards may improve individuals' compliance with an addiction recovery programs. However, individuals may be motivated to “cheat” to claim rewards by feigning compliance with the addition recovery program. Accordingly, techniques for confirming compliance prior to providing rewards to individuals is desired.

### SUMMARY

Disclosed herein are systems and methods for providing quantitative assessments of one or more individuals' risk of relapse during recovery. Various inputs, including compliance with support activities and self-care activities, are used to calculate a risk score. The risk score for one or more individuals may be provided to a case manager responsible for managing the recovery of the individuals. The quantitative assessment may allow the case manager to better prioritize the individuals under their care.

In accordance with an example described herein, a method of determining an individual in addiction recovery's risk of relapse may include receiving a plurality of inputs including GPS-verified activity check-ins for the individual, calculating a plurality of risk factors, based at least in part, on the plurality of inputs, wherein a numerical value of the plurality of risk factors is further based at least in part on a number of days the individual has been in recovery, assigning weights to individual ones of the plurality of risk factors, multiplying the plurality of risk factors by the corresponding weights assigned to generate a corresponding plurality of weighted factor values, and summing the weighted factor values to generate a risk score, wherein the risk score indicates to a case manager a quantitative assessment of the individual's risk of relapse. In some examples, the method may further include assigning sensitivity values to individual ones of the plurality of risk factors, wherein the sensitivity values are proportional to target proportions that individual ones of the plurality of risk factors contribute to the risk score, scaling the plurality of risk factors by the sensitivity values to generate a plurality of sensitized factor values, calculating differences between each of the plurality of sensitized factor values and the risk score and summing the differences to generate a delta sum, and adding the delta sum to the risk score to generate an adjusted risk score.

In accordance with an example described herein, a system for determining an individual in addiction recovery's risk of relapse may include a non-transitory computer-readable medium configured to store a plurality of inputs including GPS-verified activity check-ins for the individual, a processor communicatively coupled to the non-transitory computer-readable medium, the processor configured to: calculate a plurality of risk factors, based at least in part, on the plurality of inputs, wherein a numerical value of the plurality of risk factors is further based at least in part on a number of days the individual has been in recovery, assign weights to individual ones of the plurality of risk factors, multiply the plurality of risk factors by the corresponding weights assigned to generate a corresponding plurality of weighted factor values, and sum the weighted factor values to generate a risk score, wherein the risk score quantifies the individual's risk of relapse. The system may further include a display configured to provide the risk score to a case manager.

In accordance with an example described herein, method of triaging individuals in addiction recovery based on a risk of relapse may include receiving a plurality of inputs including GPS-verified activity check-ins for the individuals, calculating a plurality of risk factors for each individual, based at least in part, on the plurality of inputs, wherein a numerical value of the plurality of risk factors is further based at least in part on a number of days for each individual has been in recovery, assigning weights to individual ones of the plurality of risk factors, multiplying the plurality of risk factors by the corresponding weights assigned to generate a corresponding plurality of weighted factor values for each individual, summing the weighted factor values to generate a risk score for each individual, ranking the individuals from a highest priority to a lowest priority based on the risk score for each individual, wherein the highest priority individuals are at a greater risk of relapse than the lowest priority individuals. In some examples, the method may further include, assigning sensitivity values to individual ones of the plurality of risk factors, wherein the sensitivity values are proportional to target proportions that individual ones of the plurality of risk factors contribute to the risk score, scaling the plurality of risk factors by the sensitivity values to generate a plurality of sensitized factor values for each individual, calculating differences between each of the plurality of sensitized factor values and the risk score and summing the differences to generate a delta sum for each individual, adding the delta sum to the risk score to generate an adjusted risk score for each individual, and adjusting the ranking of the individuals based on the adjusted risk scores.

In accordance with an example described herein, a method of providing a reward to an individual in addiction recovery may include providing a challenge to the individual based at least in part on a number of days the individual has been in recovery, wherein the challenge includes at least one of a volume of support activities to be completed in a period of time or a volume of self-care activities to be completed in the period of time, receiving at least one of a support activity check-in rate or a self-care activity check-in rate, wherein the support activity check-in rate is based on GPS-verified check-ins for the individual, determining, based at least in part on the at least one of the support activity check-in rate or the self-care activity check-in rate, whether the challenge was completed, and if it is determined the challenge was completed, providing the reward to the individual.

### BRIEF DESCRIPTION OF THE DRAWINGS

FIG. 1 is a schematic illustration of a system arranged in accordance with examples described herein.

FIG. 2 is a block diagram of an overview for determining an individual in addiction recovery's risk of relapse in accordance with examples described herein.

FIG. 3 is a plot of a days in recovery risk curve in accordance with examples described herein.

FIG. 4 is a plot of support activity volume risk curves in accordance with examples described herein.

FIG. 5 is a plot of self-care activity volume risk curves in accordance with examples described herein.

FIG. 6 is a flow chart of a method determining an individual in addiction recovery's risk of relapse in accordance with examples described herein

FIG. 7 is a flow chart of a method for determining an adjusted risk score in accordance with examples described herein.

FIG. 8 a flow chart of a method for providing a reward to an individual in addiction recovery in accordance with examples described herein.

FIG. 9 is an illustration of an environment in which a system may operate in accordance with examples described herein.

FIG. 10 is a screenshot of an individual's information from a case manager computing device in accordance with examples described herein.

FIG. 11 is a screenshot of multiple individuals' information from a case manager computing device in accordance with examples described herein.

FIG. 12A is a screenshot of support activities to select from an individual computing device in accordance with examples described herein.

FIG. 12B is a screenshot of scheduling a self-care activity from an individual computing device in accordance with examples described herein.

FIGS. 13A-B are screenshots of a daily activity schedule from an individual computing device in accordance with examples described herein.

FIGS. 14A-C are screenshots of an incentive program from an individual computing device in accordance with examples described herein.

### DETAILED DESCRIPTION

The following description of certain embodiments is merely exemplary in nature and is in no way intended to limit the scope of the disclosure or its applications or uses. In the following detailed description of embodiments of the present systems and methods, reference is made to the accompanying drawings which form a part hereof, and which are shown by way of illustration specific embodiments in which the described systems and methods may be practiced. These embodiments are described in sufficient detail to enable those skilled in the art to practice presently disclosed systems and methods, and it is to be understood that other embodiments may be utilized and that structural and logical changes may be made without departing from the spirit and scope of the disclosure. Moreover, for the purpose of clarity, detailed descriptions of certain features will not be discussed when they would be apparent to those with skill in the art so as not to obscure the description of embodiments of the disclosure. The following detailed description is therefore not to be taken in a limiting sense, and the scope of the disclosure is defined only by the appended claims.

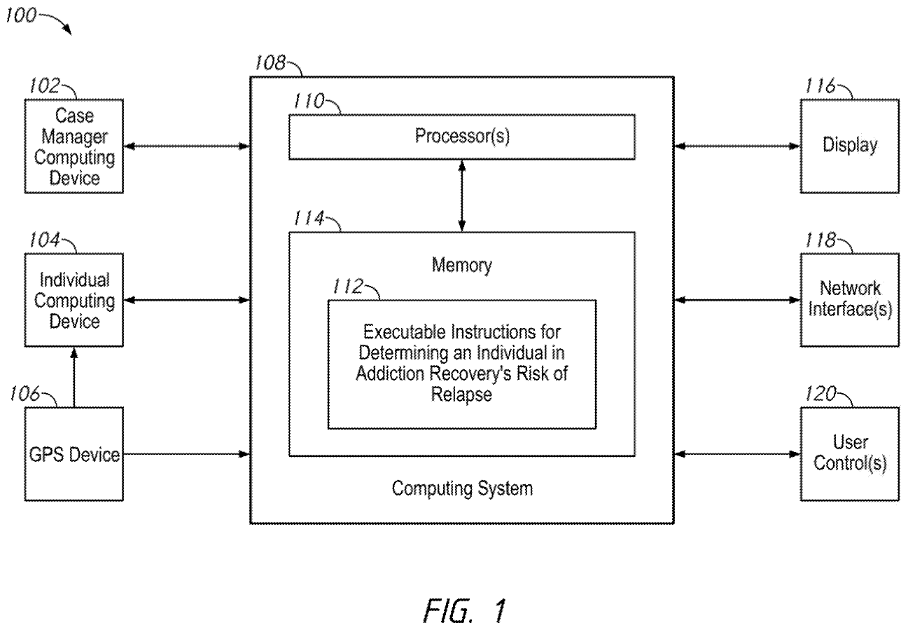

FIG. 1 is a schematic illustration of a system 100 arranged in accordance with examples described herein. The system 100 may include a case manager computing device 102, an individual computing device 104, a global positioning system (GPS) device 106, computing system 108, processor(s) 110, executable instructions for determining an individual in addiction recovery's risk of relapse 112, memory 114, display 116, and network interface(s) 118. Additional, fewer, and/or other components may be used in other examples.

Examples described herein may utilize computing systems, which may generally include hardware and/or software for implementing a model for determining an individual in addiction recovery's risk of relapse as a quantitative risk score. For example, the computing system 108 may include one or more processor(s) 110. The processor(s) 110 may be implemented for example, using one or more central processing units (CPUs), graphical processing units (GPUs), application-specific integrated circuits (ASICs), field programmable gate arrays (FPGA), or other processor circuitry. The processor(s) 110 may be in communication with memory 114. The memory 114 may generally be implemented by any computer readable media (e.g., read-only memory (ROM), random access memory (RAM), flash, solid state drive, etc.). While a single memory 114 is shown, any number may be used, and one or more memories 114 may be integrated with the processor(s) 110 in a single computing system 108 and/or located within another computing system and in communication with processor(s) 110.

In some examples, the system 100 may include display 116, which may be in communication with computing system 108 (e.g., using a wired and/or wireless connection), or the display 116 may be integrated with the computing system 108. The display 116 may display text, plots, charts, and/or other graphical information related to a risk score and/or individuals in addiction recovery (e.g., name, age, contact information). Any number or variety of displays may be present, including one or more LED, LCD, plasma, or other display devices.

In some examples, the system 100 may include network interface(s) 118. The network interface(s) 118 may provide a communication interface to any network (e.g., LAN, WAN, Internet). The network interface(s) 118 may be implemented using a wired and/or wireless interface (e.g., Wi-Fi, BlueTooth, HDMI, USB, etc.). The network interface(s) 118 may communicate data regarding the predicted efficacy of a treatment and/or recommended parameters for a treatment based OR the comparison and or statistical model implemented by the computing system 108.

In some examples, system 100 may include one or more user control(s) 120. The user control(s) may provide an interface for a user of the system 100 to provide inputs to the computing system 108. Inputs may include, but are not limited to, adjustments to the executable instructions 112, what individuals' risk scores to provide to the display 116, and the manner in which the risk scores are displayed (e.g., plot, text). The user control(s) 120 may include any type of user control, for example, a keyboard, mouse, touch pad, and/or touch screen. In some, examples, display 116 may be a touch screen and configured to include one or more soft controls that may be used to implement the user controls) 120.

In some examples, the computing system 108 may receive inputs and/or provide outputs to one or more devices such as a case manager computing device 102, an individual computing device 104, and a GPS device 106. In some examples, the case manager computing device 102 may be a mobile computing device (e.g., phone, laptop, tablet) or a stationary computing device (e.g., desktop computer). A case manager, such as someone who is responsible for managing care for one or more individuals in addiction recovery, may interact with the computing system 108 via the case manager computing, device 102. In some examples, the computing system 108 may be included on the case manager computing device 102. In some examples, display 116 may be included with the case manager computing device 102. In other examples, the case manager computing device 102 includes a separate display (not shown). In some examples, a case manager may provide inputs to the computing system 108 via the case manager computing device 102. For example, the case manager may provide individual information (e.g., support activity schedule, health records) to the computing system 108. In some examples, the computing system 108 may provide outputs to the case manager computing device 102 (e.g., risk scores, individual's contact information, treatment suggestions).

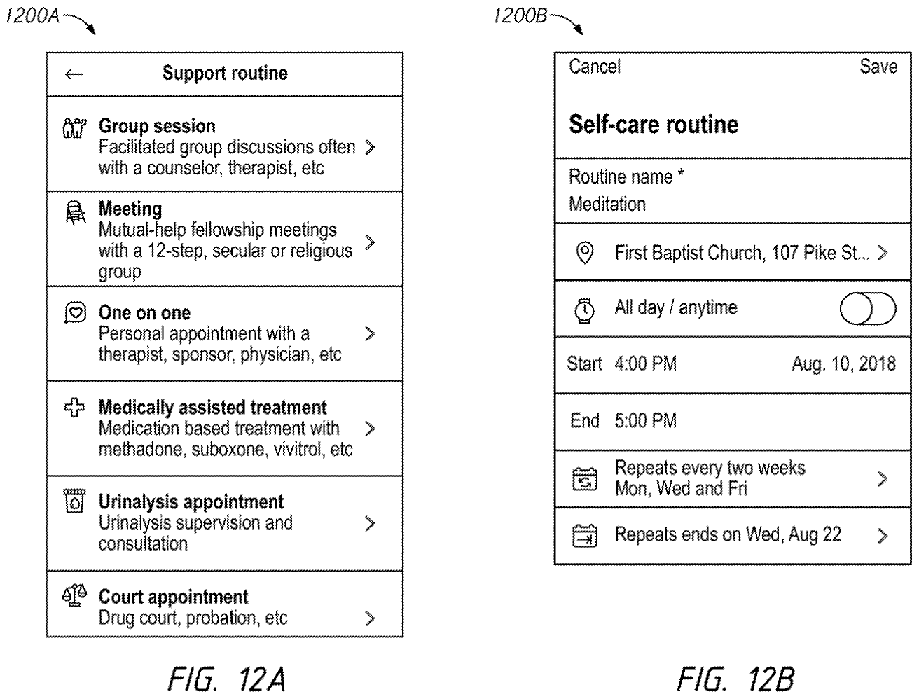

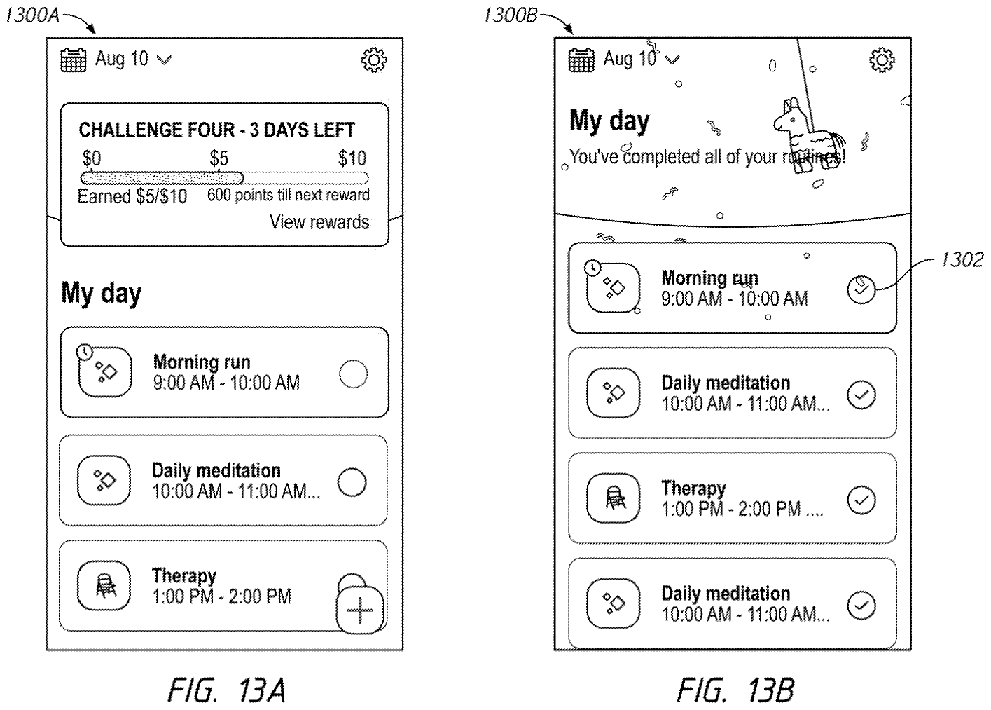

In some examples, the individual computing device 104 may be a mobile computing device (e.g., phone, laptop, tablet) or a stationary computing device (e.g., desktop computer). An individual, such as someone who is in addiction recovery (e.g., substance abuse), may interact with the computing system 108 via the individual computing device 104. For example, the computing device 104 may include an application with a user interface. In some examples, the individual may provide inputs to the computing system 108 via the individual computing device 104. For example, the individual may schedule support activities, check-in to support activities, and/or request emergency assistance (e.g., SOS request). FIG. 12A is a screenshot 1200A of support activities to select from an individual computing device in accordance with examples described herein. In sonic examples, a similar list of self-care activities to select may be provided by the individual computing device. FIG. 12B is a screenshot 1200B of scheduling a self-care activity from an individual computing device in accordance with examples described herein. In some examples, a similar scheduling tool may be provided by the individual computing device for support activities. The individual may schedule an activity and the information may be provided to the computing system 108.

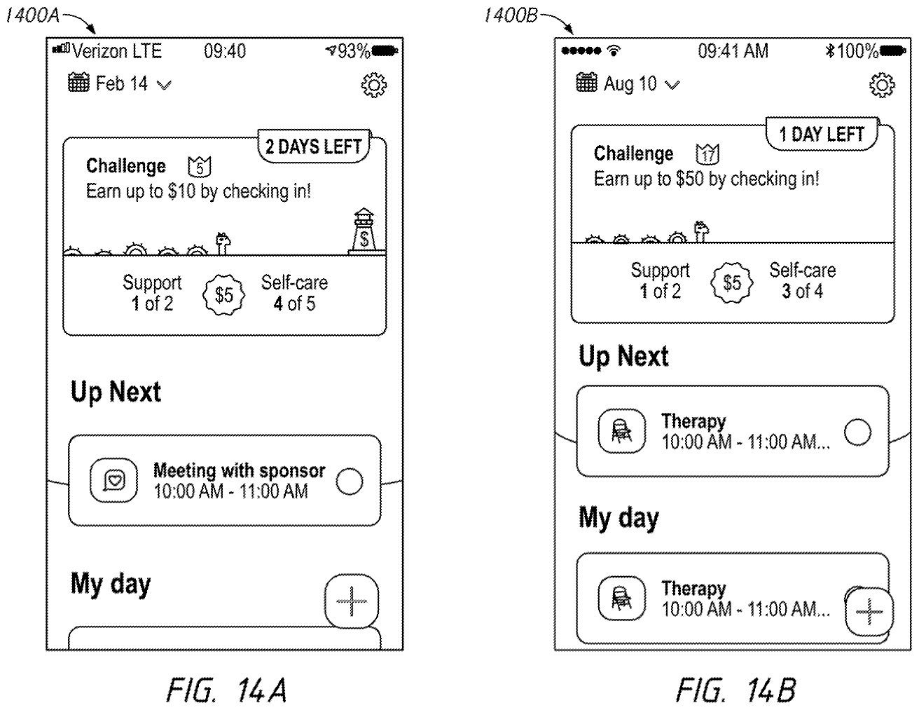

In some examples, the computing system 108 may provide outputs to the individual computing device 104 (e.g., individual's risk score, activity reminders). FIG. 13A is a screenshot 1300A of a daily activity schedule from an individual computing device in accordance with examples described herein. The computing system 108 may provide the daily activity schedule to the individual computing device 104 in some examples. As the individual checks into activities via the individual computing device 104, the computing system 108 may update the daily activity schedule. FIG. 13B is a screen shot of a daily activity schedule from an individual computing device in accordance with examples described herein. In the example shown in FIG. 13B, a visual indication 1302 is provided to indicate the individual checked into an activity. In this example, the visual indication 1302 is a checkmark, but other visual indications could be used in other examples (e.g., shading, crossing out activity).

In some examples, the GPS device 106 may be a mobile computing device (e.g., phone, laptop, tablet). In some examples, the GPS device 106 may be included with the individual computing device 104 (e.g., an individual's mobile phone may include GPS capabilities). In some examples, the GPS device 106 may be separate from the individual computing device 104. For example, the GPS device 106 may be an ankle bracelet worn by an individual in recovery configured to track the individual's movements. In another example, the GPS device 106 may be a device installed in an automobile used by the individual. In some examples, the GPS device 106 may provide inputs to the computing system 108 (e.g., a location of the GPS device 106 at a particular time). In some examples, the GPS device 106 provides location and time inputs to the individual computing device 104, which then provides the data to the computing system 108. In some examples, the computing system 108 may use inputs from the GPS device 106 to confirm inputs provided by the individual computing device 104.

In some examples, the inputs provided by devices 102, 104, and/or 106 may be stored in the memory 114. In some examples, one or more of the inputs may be used by the processor 110 when executing the executable instructions for determining an individual in addiction recovery's risk of relapse 112. The executable instructions 112 may implement a model for generating a quantitative measure of an individual's risk of relapse, referred to herein as a risk score. Thus, the inputs may be used by the processor 110 to calculate an individual's risk score.

The system 100 may be used to support individuals in addiction recovery. For example, a case manager may use system 100 to track progress of individuals under their care by confirming activity schedules and attendance at activities. A case manager may use system 100 to calculate a risk score for one or more individuals under their care. The risk score may be a quantitative measure of risk of relapse of an individual. In some examples, case manager may use the risk score to determine whether additional action is required on the part of the case manager (e.g., call to check-in with individual, schedule additional activities). In some examples, the case manager may use the risk scores of multiple individuals to rank (e.g., triage) the individuals based on the risk scores. The case manager may prioritize the individuals in terms of order or amount of time devoted to each individual in some examples. A quantitative measure of risk of relapse provided by system 100 may allow the case manager to more reliably provide a proper level of care to the individuals in their case load compared to qualitative assessments made by the case manager in some applications.

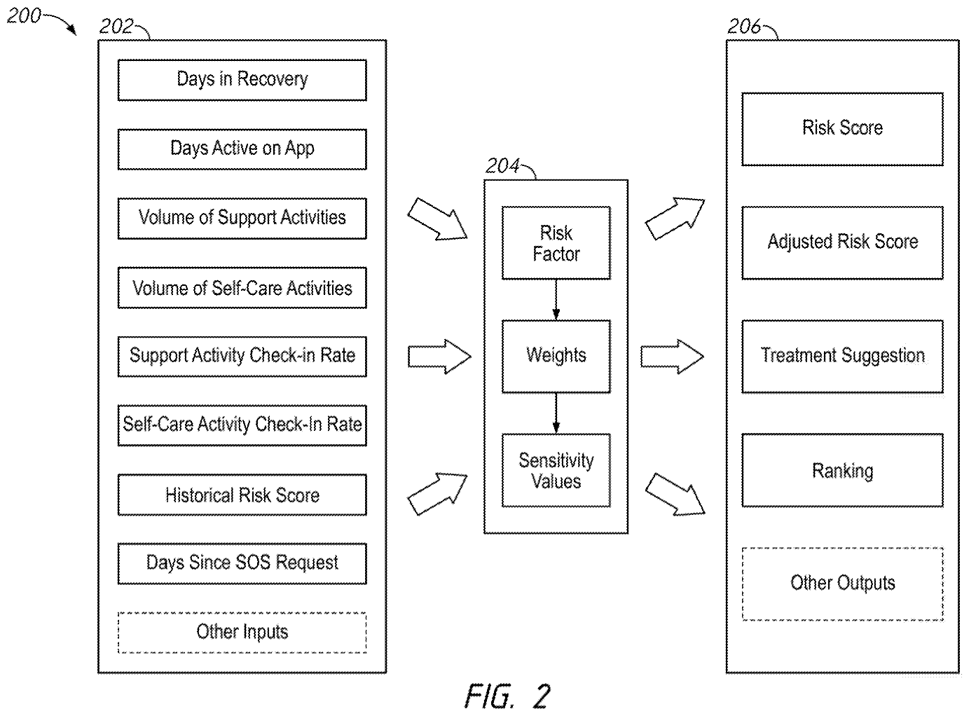

FIG. 2 is a block diagram 200 of an overview for determining an individual in addiction recovery's risk of relapse in accordance with examples described herein. In the example shown in FIG. 2, a risk model 204 may receive one or more inputs 202 and provide one or more outputs 206. The risk model 204 may be implemented by computing system 108 shown in FIG. 1 in some examples. For example, the risk model 204 may be implemented by the executable instructions 112. The one or more inputs 202 may be provided by devices 102, 104, and/or 106 shown in FIG. 1 in some examples. The one or more outputs 206 may be provided to a display, such as display 116 shown in FIG. 1, in some example. The one or more outputs 206 may also or alternatively be provided to a network interface, such as network interface 118 and/or a case manager computing device, such as case manager computing device 102 shown in FIG. 1 in some examples.

In the example shown in FIG. 2, the one or more inputs 202 may include days in recovery (Dᵣ), days active in an application (D_{w}), volume of support activities (Sᵥ), volume of self-care activities (V_{c}), support activity check-in rate (S_{c}), self-care activity check-in rate (C_{c}), historical risk score (Rₕ), and/or days since an emergency (e.g., SOS) request (D_{SOS}). Days in recovery may be a number of days an individual has been in addiction recovery without a relapse (e.g., use of an abused substance). Days active on an application may be a number of days the individual has been providing inputs to the risk model 204 and/or system 100 shown in FIG. 1. For example, the individual may provide inputs via an application on an individual device, such as individual device 104 shown in FIG. 1. In some examples, days in recovery and days active on the application may be the same.

volume of support activities may be a number of support activities scheduled for an individual during a period of time (e.g., day, week, month). Volume of self-care activities may be a number of self-care activities scheduled for an individual during a period of time (e.g., day, week, month). Support activity check-in rate may be a percentage of scheduled support activities that an individual indicates attending (e.g., check-in). In some examples, the individual may indicate attendance via an individual computing device, e.g. individual computing device 104. In some examples, a GPS device, such as GPS device 106 may verify the individual's indication of attending. In other examples, the GPS device 106 may indicate the individual's attendance at the support activity in lieu of the individual checking in. Self-care activity check-in rate may be a percentage of scheduled self-care activities that an individual indicates attending (e.g., check-in). In some examples, the individual may indicate attendance via an individual computing device, e.g. individual computing device 104.

In some examples, support activities may include activities with one or more persons providing recovery support to the individual in recovery, such as meetings with a case manager, appointments with a physician or mental health professional, and support group meetings (e.g., Alcoholics Anonymous). In some, examples, self-care activities may include activities the individual performs alone or with friends and family, such as meditation, physical activity, waking up on time, brushing teeth, etc.

In some examples, the historical risk score may be a past risk score determined for an individual by the risk model 204. The historical risk score may be an average of all past risk scores determined for an individual in some examples. In other examples, the historical risk score may be a moving average of past risk scores determined for an individual. A window for the moving average may be any period of time, for example, one week, one month, and/or one year.

In some examples, days since an SOS request may be a number of days since an individual requested emergency assistance, for example, a request via an individual computing device, such as individual computing device 104 in FIG. 1. An SOS request may be a request for an immediate check-in by a case manager, a physician, and/or mental health professional. In some examples, days since an SOS request may be omitted from the inputs.

Optionally, in some examples, other inputs may be provided to the risk model 204. For example, data relating to family history of addiction, the individual's prior history of addiction to different substances, or other medical issues may be used as inputs.

In the example shown in FIG. 2, the outputs 206 may include a risk score (R_{w}), an adjusted risk score (R_{L}), treatment suggestion, and/or a ranking. The risk score may be a quantitative measure of an individual's risk of relapse based on one or more inputs 202. The adjusted risk score may be the risk score adjusted by modifying the sensitivity of the risk model 204 to certain inputs 202, as will be described in more detail. In some examples, the risk model 204 may output treatment suggestions to a case manager based on the risk score and/or adjusted risk score calculated by the risk model 204 (e.g., contact the individual, schedule an appointment with a clinician). In other examples, the risk score and/or adjusted risk score is provided to another model and/or system for determining treatment suggestions. In some examples, the outputs 206 may include the risk scores and/or adjusted risk scores of multiple individuals. In these examples, the outputs 206 may include a ranking of individuals based on their risk scores and/or adjusted risk scores.

Optionally, in some examples, other outputs may be provided by the risk model 204. For example, data relating to how individual risk factors contributed to the risk score calculation may be provided.

In some examples, the risk model 204 may calculate numerical values for one or more of risk factors for an individual, based at least in part, on the inputs 202. A numerical value of a risk factor may be based at least in part on a number of days the individual has been in recovery. The risk model 204 may assign weights to the risk factors. Each of the risk factors may have different weights or some of the risk factors may be weighted the same in some examples. The risk model 204 multiplies the risk factors by the corresponding weights assigned to generate a corresponding set of weighted factor values and sums the weighted factor values to generate a risk score, which quantifies the individual's risk of relapse.

In the examples where the risk model 204 provides an adjusted risk score, the risk model 204 may assign sensitivity values to the risk factors. The sensitivity values may be proportional to target proportions that individual ones of the risk factors contribute to the risk score. The risk factors may be scaled by the sensitivity values to generate a plurality of sensitized factor values. Differences between each of the plurality of sensitized factor values and the risk score may be summed to generate a delta sum. The delta sum may be added to the risk score to generate an adjusted risk score.

In some examples, the risk factors, may include risk from days in recovery (R_{d}), risk from support activity check-in rate (R_{sc}), risk from self-care activity check-in rate (R_{cc}), risk from support activity volume (Rₛᵥ), risk from self-care activity volume (R_{cv}), and historical risk (Rₕ).

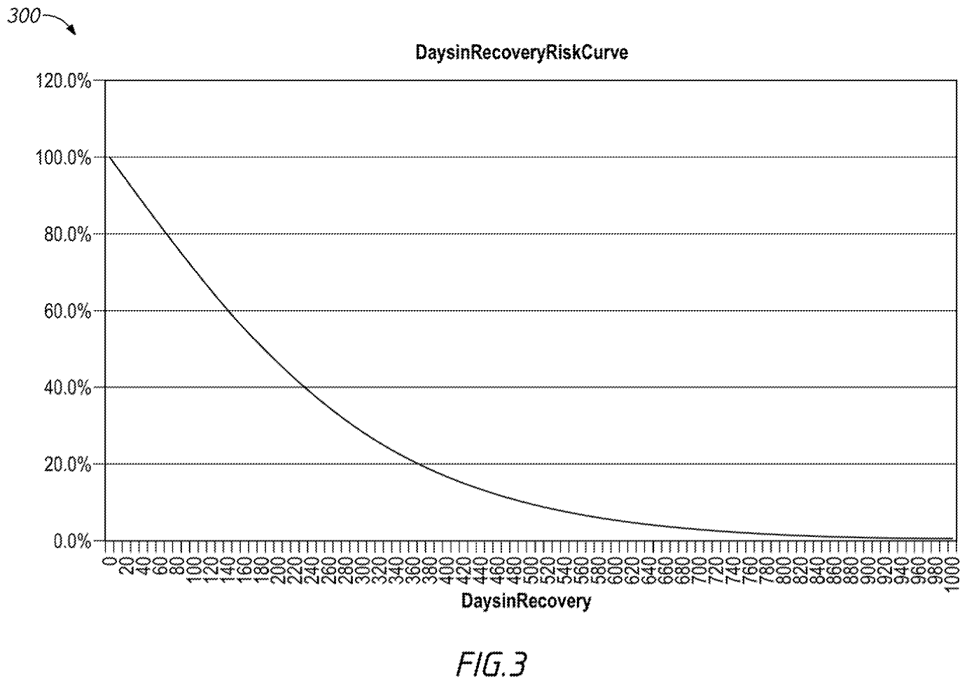

An example implementation of the risk model 204 will now be described. As mentioned previously, the risk model calculates values fir a variety of risk factors based on one or more inputs 202. Risk from days in recovery may be calculated as:

$${R}_{d} = \frac{2}{1 + {e}^{0.006 \cdot {D}_{r}}} \qquad ( 1 )$$

where Dᵣ is the input 202, days in recovery. From Equation 1, the risk from days in recovery is inversely proportional to the number of days in recovery. That is, the longer an individual is in recovery (e.g., days since the last relapse), the lower the individual's risk of a relapse. An example days in recovery risk curve is shown in plot 300 of FIG. 3. As shown in plot 300, the risk from days in recovery decays exponentially over time. In other implementations of the risk model 204, the exponential curve may have a different exponent. For example, different substances may have different “tipping points” or rates at which the risk of relapse decreases over time.

Risk from support activity check-in rate may be calculated as:

R_{sc}=1−S_{c} (2)

where S_{c} is the input 202 support activity check-in rate. Similarly, risk from self-care activity check-in rate may be calculated as:

R_{cc}=1−C_{c} (3)

where C, is the input 202 self-care activity check-in rate. For both Equations 2 and 3, the risks associated with the support activity and self-care activity check-in rates decrease as the rate of check-in increases. That is, an individual that attends most or all of scheduled activities is at a lower risk of relapse than an individual that fails to attend most or all of scheduled activities.

Risk from support activity volume may vary depending on the number of days an individual has been in recovery. For example, an individual may be expected to engage in a larger number of support activities early in recovery and fewer activities later in recovery, Continuing this example, an individual in a first year of recovery may be considered higher risk for having a low volume of support activities whereas an individual several years into recovery may be considered low risk for the same volume of support activities. In some examples, a set number of activities based on a number of days in recovery with set risk thresholds may be used to calculate risk from support activity volume. In some examples, the expected number of activities E(nₛ) may be defined by a function dependent on the number of days in recovery. In some examples, E(nₛ) may be a nonlinear function. In an example implementation, the risk from support activity volume ma be calculated as:

$${R}_{sv} = \{ \begin{matrix}\begin{matrix}\frac{0.9}{1 + {e}^{{( {S}_{v} - E ( {S}_{v 3} ) )}^{3}}} + \frac{1}{1 + {e}^{{( {S}_{v} - E ( {S}_{v 0} ) )}^{1.5}}} - \\ {R}_{d} \cdot \frac{0.9}{1 + {e}^{{( {S}_{v} - E ( {S}_{v 3} ) )}^{3}}}\end{matrix} & for {D}_{w} \geq 7 \\ \begin{matrix}\frac{0.9}{1 + {e}^{{( \frac{7 \cdot {S}_{v}}{{D}_{w}} - E ( {S}_{v 3} ) )}^{3}}} + \frac{1}{1 + {e}^{{( \frac{7 \cdot {S}_{v}}{{D}_{w}} - E ( {S}_{v 0} ) )}^{1.5}}} - \\ {R}_{d} \cdot \frac{0.9}{1 + {e}^{{( \frac{7 \cdot {S}_{v}}{{D}_{w}} - E ( {S}_{v 3} ) )}^{3}}}\end{matrix} & for {D}_{w} < 7\end{matrix} \qquad ( 4 )$$

where Sᵥ is the support activity volume in a period of time, E(Sᵥ₀) is the expected weekly volume of support activities at recovery start and E(Sᵥ₃) is the expected weekly volume of support activities after three years in recovery. In some implementations, E(Sᵥ₀) may be 4 and E(Sᵥ₃) may be 2.5, but other values may be used in other implementations. D_{w} is the number of days active on the application. D_{w} may be used to adjust the equation for Rₛᵥ when an individual has been providing inputs into the risk model 204 for less than the period of time for Sᵥ. In the example implementation shown in Equation 4, the period of time is seven days (e.g., one week). In other implementations, the period of time may be different (e.g., two weeks, one month).

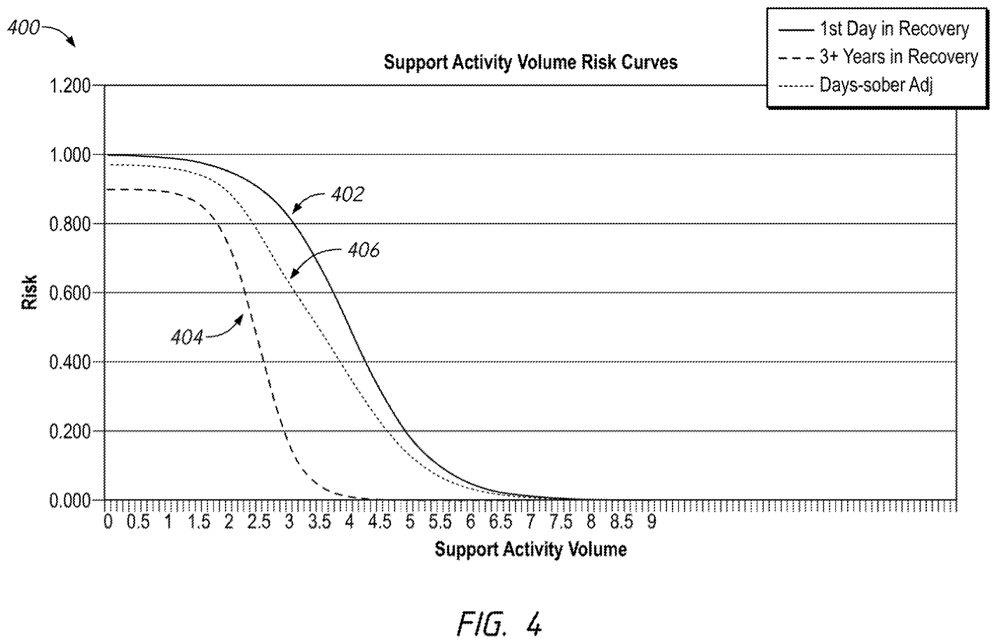

Example support activity volume risk curves are shown in plot 400 of FIG. 4. Curve 402 is an example of a risk curve for an individual on a first day of recovery. Curve 404 is an example of a risk curve for an individual three years into recovery. Curve 406 is an example of a risk curve for an individual 100 days into recovery. As demonstrated by the curves in plot 400, as an individual spends more time in recovery, fewer support activities are required to reduce the risk of relapse.

Similarly, risk from self-care activity volume may vary depending on the number of days an individual has been in recovery. An individual early in recovery may require more self-care activities to maintain a low risk of relapse than an individual further along in recovery. In some examples, a set number of activities based on a number of days in recovery with set risk thresholds may be used to calculate risk from self-care activity volume. In some examples, the expected number of activities E(n_{c}) may be defined by a function dependent on the number of days in recovery. In some examples, E(n_{c}) may be a nonlinear function. In an example implementation, the risk from self-care activity volume may be calculated as:

$${R}_{cv} = \{ \begin{matrix}\begin{matrix}\frac{0.9}{1 + {e}^{{( {C}_{v} - E ( {C}_{v 3} ) )}^{3}}} + \frac{1}{1 + {e}^{{( {C}_{v} - E ( {C}_{v 0} ) )}^{1.5}}} - \\ {R}_{d} \cdot \frac{0.9}{1 + {e}^{{( {C}_{v} - E ( {C}_{v 3} ) )}^{3}}}\end{matrix} & for {D}_{w} \geq 7 \\ \begin{matrix}\frac{0.9}{1 + {e}^{{( \frac{7 \cdot {C}_{v}}{{D}_{w}} - E ( {C}_{v 3} ) )}^{3}}} + \frac{1}{1 + {e}^{{( \frac{7 \cdot {C}_{v}}{{D}_{w}} - E ( {C}_{v 0} ) )}^{1.5}}} - \\ {R}_{d} \cdot \frac{0.9}{1 + {e}^{{( \frac{7 \cdot {C}_{v}}{{D}_{w}} - E ( {C}_{v 3} ) )}^{3}}}\end{matrix} & for {D}_{w} < 7\end{matrix} \qquad ( 5 )$$

where Cᵥ is the self-care activity volume, in a period of time, E(Cᵥ₀) is the expected weekly volume of self-care activities at recovery start and E(Cᵥ₃) is the expected weekly volume of self-care activities after three years in recovery. In some implementations, E(Sᵥ₀) may be 8 and E(Cᵥ₃) may be 5, but other values may be used in other implementations. Similar to Equation 4, D_{w} may be used to adjust the equation for R_{cv} when an individual has been providing inputs into the risk model 204 for less than the period of time for Cᵥ. In the example implementation shown in Equation 5, the period of time is seven days (e.g., one week). In other implementations, the period of time may be different (e.g., two weeks, one month).

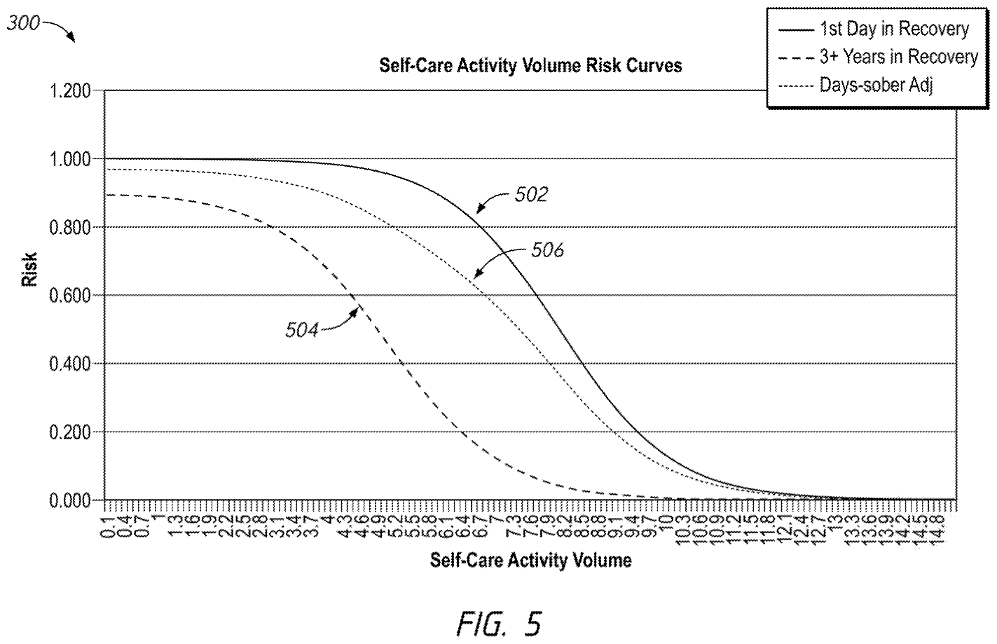

Example self-care activity volume risk curves are shown in plot 500 of FIG. 5. Curve 502 is an example of a risk curve for an individual on a first day of recovery. Curve 504 is an example of a risk curve for an individual three years into recovery. Curve 506 is an example of a risk curve for an individual 100 days into recovery. As demonstrated by the curves in plot 500, as an individual spends more time in recovery, fewer self-care activities are required to reduce the risk of relapse. Furthermore, as can be seen in comparing plots 400 and 500, the rate of risk reduction over time may be different between self-care activity volume, and support activity volume in some examples. In some examples, more self-care activities compared to support activities may be expected, but in other examples, fewer self-care activities compared to support activities may be expected. In other examples, the number of self-care activities compared to the number of support activities may change depending on a number of days in recovery.

In the example implementation described, the risks from support and self-care activity volumes are based at least in part on the number of days in recovery. However, in other implementations, additional inputs may be used to calculate the risk from support and self-care activity volumes (e.g., type of substance abused, family history of addiction, other health conditions).

The historical risk score may be calculated as:

$${R}_{h} = \frac{{\sum }_{n = 1}^{28} {T}_{n} \cdot ( 1 - \frac{n}{29} )}{100 \cdot {\sum }_{n = 1}^{28} ( 1 - \frac{n}{29} )} \qquad ( 6 )$$

where Tₙ is the risk score from the n^{th} day prior. In the example implementation, the historical risk is, based an individual's risk scores over the, past 28 days. However, the historical risk could be based on risk scores over a shorter or longer period of time (e.g., one week, one year). In some examples, the historical risk score may be an average of all past risk scores determined for an individual in some examples. In other examples, the historical risk score may be a moving average of past risk scores determined for an individual. A window for the moving average may be any period of time, for example, one week, one month, and/or one year.

The risk factors may be assigned weights. The weight for a risk factor may be proportional to how significant the risk factor is in determining a risk of relapse. In some examples, the calculated risk factors may not equally contribute to an individual's risk of relapse. In some implementations, the number of days in recovery may be the most significant risk factor for relapse. In some implementations, the check-in rate for support activities, which may be verified by GPS, may be more significant than the check-in rate for self-care activities. In some implementations, the volume of activities (support or self-care) may be less significant than the check-in rate for those activities. Each risk factor may be multiplied by its assigned weight. The risk score may be calculated as:

$${R}_{w} = \frac{{R}_{d} \cdot {W}_{d} + {R}_{sc} \cdot {W}_{sc} + {R}_{cc} \cdot {W}_{cc} + {R}_{sv} \cdot {W}_{sv} + {R}_{cv} \cdot {W}_{cv} + {R}_{h} \cdot {W}_{h}}{{W}_{d} + {W}_{sc} + {W}_{cc} + {W}_{sv} + {W}_{cv} + {W}_{h}} \qquad ( 7 )$$

Where Wₓ is the weight assigned to a risk factor Rₓ. In an example implementation, W_{d} may be 10, W_{sc} may be 8, W_{cc} may be 7, Wₛᵥ may be 6, W_{cv} may be 6, and Wₕ may be 10. However, other weights may be used in other implementations. In some implementations, a case manager may set and/or adjust the weights. For example, the case manager may provide inputs via a case manager computing device and/or a user control of a computing system. For example, the case manager computing device 102 and user control 120 shown in FIG. 1. The higher the risk score, the higher the likelihood that the individual will suffer a relapse.

While the example implementation described includes risk from days in recovery (R_{d}), risk from support activity check-in rate (R_{sc}), risk from self-care activity check-in rate (R_{cc}), risk from support activity volume (Rₛᵥ), risk from self-care activity volume (R_{cv}), and historical risk (Rₕ), in other examples, the risk model 204 may calculate fewer, different, and/or additional risk factors. In some examples, different or additional risk factors may be based on other inputs. For example, the risk model 204 may calculate a risk factor based on a number of substances abused by an individual. In another example, the risk model 204 may calculate a risk factor based on a number of mental health incidents and time since those mental health incidents suffered by the individual. These different and/or additional risk factors may also be assigned weights for calculating the risk score.

In some examples, the risk score may be provided to a case manager (e.g., via a display). The weights may also be provided to the case manager in some examples, which may assist the case manager in understanding how the risk score was determined by the risk model 204. When the risk model 204 calculates the risk scores for multiple individuals, the risk model 204 may provide a ranking of the individuals from high priority to low priority. Individuals with higher scores may be higher priority while individuals with lower scores may be lower priority. This may assist the case manager in determining how to allocate their limited time effectively.

As mentioned previously, the risk model 204 may calculate an adjusted risk score (R_{L}). The risk model 204 may adjust the sensitivity of the risk score to the various risk factors. This may reduce false high risk scores ardor low risk scores. For example, an individual who has been in recovery for a long period of time (e.g., a long period of time since a relapse) may fail to check-in to several support activities in a week. However, the risk model 204 may overreact to the individual's failure to check-in and determine a high risk score. This may cause a case manager to over prioritize the individual, possibly at the expense of other individuals at higher risk of relapse.

Continuing with the example implementation of the risk model 204, the risk model may define an unsealed sensitivity function as:

$$v ( r ) = \{ \begin{matrix}\frac{- 0.1}{r - 1.1} - 0.1 & for r \geq 0.1 , \\ 0 & for 0 \leq r < 0.1\end{matrix} \qquad ( 8 )$$

where r is one of the individual risk factors R_{d}, R_{sc}, R_{cc}, Rₛᵥ, R_{cv}, Rₕ). The unsealed sensitivity function receives a risk factor as an input and outputs an unsealed sensitivity. Equation 8 is only an example of an unsealed sensitivity function. In other implementations, other functions may be used.

The risk model 204 may define a scaled sensitivity function as:

$$t ( v ) = \{ \begin{matrix}v ( r ) \cdot V & for v ( r ) \cdot {V}_{r} < M , \\ M & for v ( r ) \cdot {V}_{r} \geq M\end{matrix} \qquad ( 9 )$$

where V is a sensitivity value (e.g., scalar) assigned to a risk factor r and M is the maximum value allowed for V. In an example implementation, M may be 0.9. The scaled sensitivity function of Equation 9 accepts an unsealed sensitivity (e.g., the output of Equation 8) and outputs a sensitized factor value (e.g., scaled sensitivity). In some examples, sensitivity values for the historical risk score and/or days in recovery may be set at or near the maximum to prevent local temporal deviations in compliance with scheduling, and/or check-ins for activities. On the other hand, setting sensitivity values high for these risk factors may amplify the risk for individuals early in recovery and/or a poor track record for checking-in. For these individuals, minor changes in check-in rate and/or activity volume may warrant attention by a case manager. In some implementations, the case manager may adjust the sensitivity values applied by the risk model 204. For example, the case manager may provide inputs via a case manager computing device and/or a user control of a computing system. For example, the case manager computing device 102 and user control 120 shown in FIG. 1.

Based on the risk score R_{w}, the unsealed sensitivity function, and the scaled sensitivity function, the risk model 204 may calculate a scaled-sensitivity adjusted risk delta as:

$$f ( t ) = \{ \begin{matrix}{R}_{w} \cdot [ 1 - t ( v ( r ) ) ] + 100 \cdot r \cdot t ( v ( r ) ) & \begin{matrix}for {R}_{w} \cdot [ 1 - t ( v ( r ) ) ] + \\ 100 \cdot r \cdot t ( v ( r ) ) > 0\end{matrix} \\ 0 & otherwise\end{matrix} \qquad ( 10 )$$

Thus, a difference between the significance of the risk factor to the risk score and the influence based on the sensitivity value are calculated fur each risk factor. The sum of the deltas (e.g., differences) is then calculated as:

$${V}_{T} = {\sum }_{r} f ( t ( v ( r ) ) ) \qquad ( 11 )$$

The delta sum is then added to the risk score to determine an adjusted risk score:

$${R}_{L} = \{ \begin{matrix}{R}_{w} + {V}_{T} & for {R}_{w} + {V}_{T} \leq 100 \\ 100 & otherwise\end{matrix} \qquad ( 12 )$$

In the example implementation shown in Equation 12, the maximum adjusted risk score is 100. However, in other implementations, adjusted risk scores over 100 may be permitted. In some implementations, when an individual makes an SOS request, the risk score and/or the adjusted risk score may automatically be set to 100 or other value set as the maximum risk score.

Thus, as described and shown in FIG. 2, the risk model 204 may provide a quantitative measure of risk of relapse of an individual in addiction recovery.

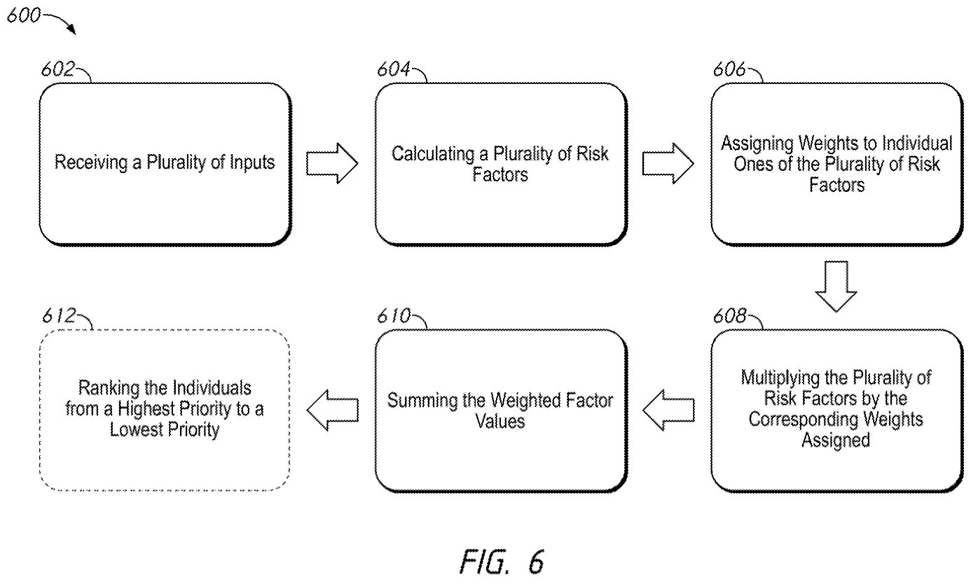

FIG. 6 is a flow chart 600 that provides an overview of a method determining an individual in addiction recovery's risk of relapse in accordance with examples described herein. The method shown in flow chart 600 may be implemented by the risk model 204 and/or system 100 shown in FIG. 1 in some examples.

At block 602, a step of “receiving a plurality of inputs” may be performed. In some examples, the inputs may be received by a computing system, such as computing system 108 shown in FIG. 1. The inputs may include GPS-verified activity check-ins for the individual in some examples. In some examples, the inputs may include the number of days in recovery, a volume of support activities, a volume of self-care activities, a historical risk score a support activity check-in rate, and a self-care activity check-in rate. In some examples, the support activity check-in rate and the self-care activity check-in rate are based, at least in part, on the GPS-verified activity check-ins. In some examples, the inputs further includes a number of days since the individual made an SOS request. In some examples, one or more inputs may be provided by an individual via an individual computing device such as individual computing device 104 shown in FIG. 1. In some examples, the GPS-verification may be provided by a GPS device, such as GPS device 106 shown in FIG. 1.

At block 604, a step of “calculating a plurality of risk factors” may be performed. In some examples, the calculating may be performed by the computing system 108 shown in FIG. 1. In some examples, the calculating may be performed by the processor(s) 110 of the computing system 108. In some examples, a numerical value of the plurality of risk factors is further based at least in part on a number of days the individual has been in recovery. In some examples, a risk factor associated with the number of days in recovery decreases as the number of days in recovery increases. In some examples, the risk factor associated with the number of days in recovery decreases exponentially. In some examples, a risk factor associated with the support activity volume is based on a number of support activities per week and the number of days in recovery and a risk factor associated with the self-care activity volume is based on a number of self-care activities per week and the number of days in recovery. In some examples, the number of support activities per week required to generate a low value for the risk, factor decreases as the number of days in recovery increases, and the number, of self-care activities per week required to generate a low value for the risk factor decreases as the number of days in recovery increases. In some examples, a risk factor associated with the support activity check-in rate increases as the support activity check-in rate decreases and wherein a risk factor associated with the self-care activity check-in rate increases as the self-care activity check-in rate decreases. In some examples, a risk factor associated with the historical risk score is proportional to the historical risk score.

At block 606, a step of “assigning weights to individual ones of the plurality of risk factors” may be performed. In some examples, the assigning may be performed by the computing system 108 shown in FIG. 1. In some examples, the assigning may be performed by the processor(s) 110 of the computing system 108. In some examples, a risk factor associated with the number of days in recovery is assigned a highest weight of the weights. In some examples, a risk factor associated with the historical risk score and a risk factor associated with the number of days in recovery are assigned equal weights. In some examples, a weight assigned to a risk factor associated with the support activity check-in rate is greater than a weight assigned to a risk factor associated with the self-care activity check-in rate.

At block 608, a step of “multiplying the plurality of risk factors by the corresponding weights assigned” is performed. In some examples, the multiplying may be performed by the computing system 108 shown in FIG. 1. In some examples, the multiplying may be performed by the processor(s) 110 of the computing system 108. The multiplication may generate a corresponding plurality of weighted factor values. At block 610, a step of “summing the weighted factor values” may be performed. In some examples, the summing may be performed by the computing system 108 shown in FIG. 1. In some examples, the summing may be performed by the processor(s) 110 of the computing system 108. Summing the weighted factor values may generate a risk score. The risk score may indicate to a case manager a quantitative assessment of the individual's risk of relapse. In some examples, the risk score may be provided by the computing system 108 to the case manager computing device 102 shown in FIG. 1. In some examples, the risk score may be provided by the computing system 108 to the display 116.

In a method where a risk score is calculated for multiple individuals, at block 612, a step of “ranking the individuals from a highest priority to a lowest priority” may be performed. In some examples, the ranking may be performed by the computing system 108 shown in FIG. 1. In some examples, the ranking may be performed by the processor(s) 110 of the computing system 108. The ranking may be based on the risk score for each individual where the highest priority individuals are at a greater risk of relapse than the lowest priority individuals. In some implementations, individuals with higher risk scores are at greater risk than those with lower risk scores.

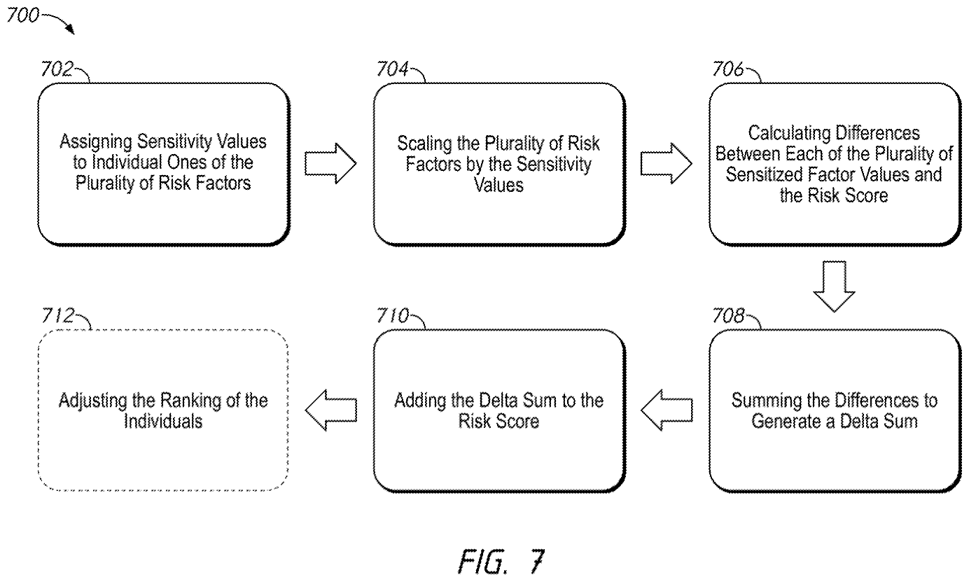

FIG. 7 is a flow chart 700 that provides an overview of a method for determining an adjusted risk score in accordance with examples described herein. The method shown in flow chart 700 may be implemented by the risk model 204 and/or system 100 shown in FIG. 1 in some examples. In some examples, the blocks in flow chart 700 may be performed after the blocks of flow chart 600 have been performed.

At block 702, a step of “assigning sensitivity values to individual ones of the plurality of risk factors” may be performed. In some examples, the assigning may be performed by the computing system 108 shown in FIG. 1. In some examples, the assigning may be performed by the processor(s) 110 of the computing system 108. The sensitivity values may be proportional to target proportions that individual ones of the plurality of risk factors contribute to the risk score in some examples.

At block 704, a step of “scaling the plurality of risk factors by the sensitivity values” may be performed. In some examples, the scaling may be performed by the computing system 108 shown in FIG. 1. In some examples, the scaling may be performed by the processor(s) 110 of the computing system 108. Scaling the risk factors may generate a plurality of sensitized factor values. At block 706, a step of “calculating differences between each of the plurality of sensitized factor values and the risk score” may be performed. In some examples, the calculating may be performed by the computing system 108 shown in FIG. 1. In some examples, the calculating may be performed by the processor(s) 110 of the computing system 108. At block 708, a step of “summing the differences to generate a delta sum” may be performed. In some examples, the summing may be performed by the computing system 108 shown in FIG. 1. In some examples, the summing may be performed by the processor(s) 110 of the computing system 108. At block 710, a step of “adding the delta sum to the risk score” may be performed. In some examples, the adding may be performed by the computing system 108 shown in FIG. 1. In some examples, the adding may be performed by the processor(s) 110 of the computing system 108. Adding the delta sum to the risk score may generate an adjusted risk score.

In a method where an adjusted risk score is calculated for multiple individuals, at block 712, a step of “adjusting the ranking of the individuals” may be performed. In some examples, the adjusting may be performed by the computing system 108 shown in FIG. 1. In some examples, the adjusting may be performed by the processor(s) 110 of the computing system 108. The adjustment may be based on the adjusted risk scores.

In the examples discussed with reference to FIGS. 6 and 7, the risk scores, adjusted risk scores and/or rankings may be provided on a display. The display may be associated with a computing system implementing a risk model, such as system 100 shown in FIG. 1 and/or with a case manager computing device, such as case manager computing device 102 shown in FIG. 1. In some examples, individuals may be provided their own risk scores and/or adjusted risk scores on an individual computing device, such as individual computing device 104 shown in FIG. 1.

An incentive program may be used encourage individuals in addiction recovery to remain in recovery. In some examples, the incentive program may provide rewards to the individuals. The rewards may be based on an individual's compliance with addiction recovery to encourage continued compliance. Rewards may be monetary (e.g., cash, gift cards, discounts) and/or non-monetary (e.g., event, food). In some examples, individuals may earn rewards by completing challenges. Challenges may include one or more tasks that the individual must complete to earn the reward. In some examples, a challenge may require an individual to check in to a certain number of support activities and/or a certain number of self-care activities within a period of time (e.g., three days, one week). In some examples, a challenge may require an individual to check in to a certain type of support activity and/or self-care activity within a period of time (e.g., support group meeting, yoga class). In some examples, the tasks included in a challenge may be based, at least in part, on a number of days the individual has been in recovery. In some examples, the type and/or amount of the reward may be based, at least in part, on the number of days the individual has been in recovery.

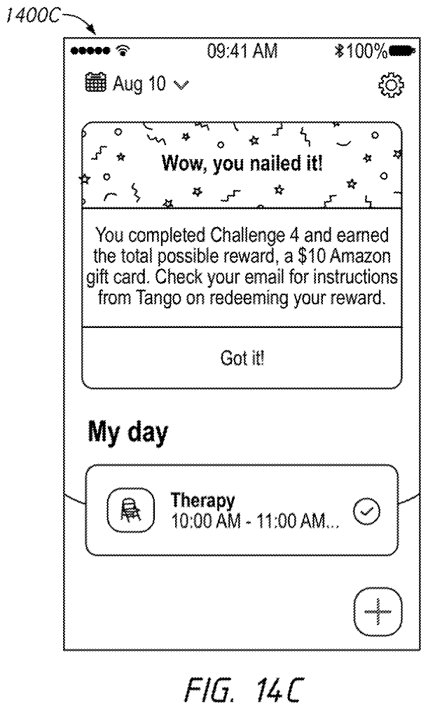

FIGS. 14A-C are screenshots of an incentive program from an individual computing device in accordance with examples described herein. In some examples, the incentive program may be administered, at least in part, on an individual computing device (e.g., individual computing device 106). FIG. 14A is an example screenshot 1400A of a challenge that requires checking into two support activities and five self-care activities within a certain period of time. If the individual completes the challenge, the individual will receive a ten dollar reward. FIG. 14B is an example screenshot 1400B of a challenge that requires checking into 2 support activities and four self-care activities within a certain period of time. If the individual completes the challenge, the individual will receive a fifty dollar reward. In some examples, the challenge shown in screenshot 1400A may be provided to an individual in early stages of recovery and the challenge shown in screenshot 1400B may be provided to an individual in later stages of recovery (e.g., more days in recovery than an individual in the early stages of recovery). In other examples, the challenge shown in screenshot 1400A may be provided to an individual in later stages of recovery and the challenge shown in screenshot 1400B may be provided to an individual in earl stages of recovery.

If the individual completes the one or more tasks of the challenge, the individual earns the reward. FIG. 14C is an example screenshot 1400C of a notification to an individual that a challenge has been completed and a reward has been earned. In sonic examples, completion of the challenge may be reported to a case manager who provides the reward to the individual (e.g., during a regularly scheduled meeting). In some examples, a computing system may provide the reward to the individual via an individual computing device (e.g., an e-gift card via an application on a mobile device). While rewards may incentivize individuals to remain in recovery, the rewards may also incentivize individuals to feign compliance with addiction recovery in order to receive the rewards. In some examples, UPS verified check-ins may be used to reduce the risk of providing rewards to individuals that feign compliance.

In some examples, a system used for determining an individual in addiction recovery's risk of relapse may also be used to administer the incentive program. For example, system 100 shown in FIG. 1 may be used. In some examples, memory 114 may further include executable instructions for administering an incentive program for an individual in addiction recovery. The executable instructions may be executed by processor 110. In some examples, the incentive program may use one or more of the same inputs 202 used by the risk model 204 to provide rewards. For example, the incentive program may use days in recovery, volume of support activities, volume of self-care activities, support activity, check-in rate, and/or self-care activity check-in rate to determine challenges and/or provide rewards. In other examples, a separate system and inputs may be used.

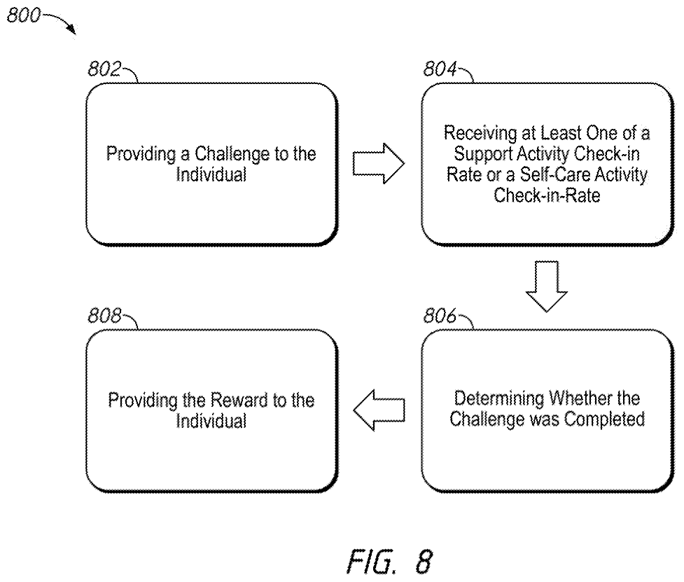

FIG. 8 is a flow chart 800 of a method of providing a reward to an individual in addiction recovery in accordance with examples provided herein. In some examples, the method may be performed by a computing system, such as computing system 108 shown in FIG. 1. In some examples, the method may be performed by a processor included in the computing device, such as processor 110 shown in FIG. 1.

At block 802, a step of “providing a challenge to the individual” may be performed. The challenge may be based at least in part on a number of days the individual has been in recovery. The challenge may include a volume of support activities to be completed in a period of time and/or a volume of self-care activities to be completed in the period of time. The challenge may be provided by a computing system, such as computing system 108 shown in FIG. 1 to an individual computing device, such as individual computing device 104 shown in FIG. 1.

At block 804, a step of “receiving at least one of a support activity check-in rate or a self-care activity check-in rate” may be performed. In some examples, the support activity check-in rate is based on UPS-verified check-ins for the individual. The check-in rates may be based on check-ins provided to the computing system 108 by an individual via individual computing device 104 in some examples. In some examples, the check-ins may be GPS-verified by GPS device 106 shown in FIG. 1.

At block 806, a step of “determining whether the challenge was completed” may be performed. The determination may be performed by the computing system 108 in some examples. The determination may be based at least in part on the at least one of the support activity check-in rate or the self-care activity check-in rate. If it is determined the challenge was completed, at block 808, a step of “providing the reward to the individual” may be performed. In some examples, the computing system 108 may provide the reward to the individual via the individual computing device 104 (e.g., a digital gift card). In other examples, providing the reward to the individual may include, sending a notification to a case manager that the individual has completed the challenge. For example, the computing system 108 may provide the notice to the case manager via a case manager computing device 102 shown in FIG. 1. The case manager may then provide the reward to the individual.

In some implementations, the incentive program may provide inputs to the risk model 204. For example, whether or not an individual has successfully completed a challenge may be used to calculate one or more risk factors.

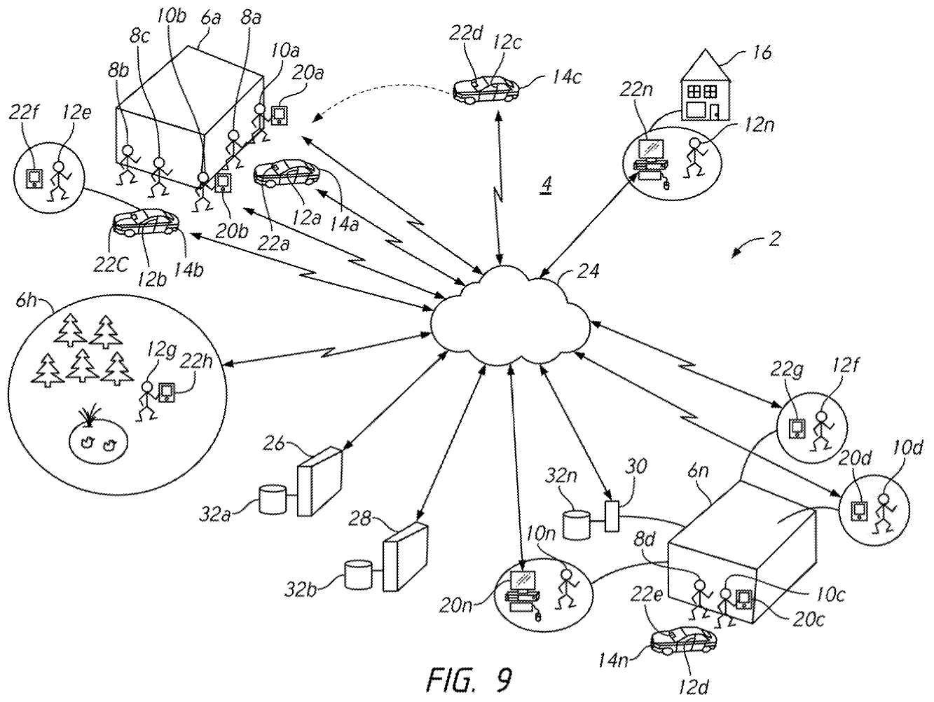

FIG. 9 illustrates an environment 4 in which a system 2 may operate to determine risk of relapse of individuals in recovery and/or administer an incentive program in accordance with examples described herein. In some examples, system 2 may include system 100 shown in FIG. 1. The environment 4 can include a number of facilities 6a (collectively 6a; only one shown), a number of non-connected individuals 8a-8n (collectively 8; only four shown), a number of case managers 10a-n On (collectively 10; only five shown), a number of connected individuals 12a-12n. (collectively 12; only eight shown), a number of vehicles 14a-14n (collectively 14; only four shown), and a residence 16 (only one shown),

A first facility 6a can be an in-patient rehabilitation treatment center. A second facility 6b can be a public park. A third facility 6n can host at least one addiction recovery meeting.

The individuals 8, 12 have at least one respective addiction to at least one respective activity, behavior, item, or substance. For example, a given one of the individuals 8, 12 may have at least one addiction to at least one drug, alcohol, sex, gambling, theft, technology, lying, gaming, an eating disorder, food, exercise, etc.

The system 2 comprises at least one individual computing device 22a-22n (collectively 22; only eight shown) of at least a respective one of the individuals 12. In some examples, individual computing devices 22 may include individual computing device 104 shown in FIG. 1. The system 2 can also comprise at least one case manager computing device 20a-20n (collectively 20; only five shown) of at least a respective one of the case managers 10. In some examples, case manager computing devices 20 may include case manager computing device 102 shown in FIG. 1. The system 2 also comprises at least one recovery server 28. In some examples, recovery server 28 may include computing system 108 shown in FIG. 1.

The case manager computing devices 20 can, for example, take the form of any variety of communications enabled devices, including wireless communications devices such as tablet computers, smartphones, dudphones (e.g., a phone that, as opposed to a smartphone, permits limited operation such as only one or more operation explained herein), netbook computers with wireless modems, wireless enabled head units of vehicles, wearable devices (e.g., bracelet, anklet, watch, necklace, glasses, headband, in-ear device), and implantable devices (e.g. subcutaneous implanted device). The individual computing devices 22 can, for example, take the form of any variety of communications enabled devices, including wireless communications devices such as tablet computers, smartphones, dudphones, netbook computers with wireless modems, wireless enabled head units of vehicles, wearable devices (e.g., bracelet, anklet, watch, necklace, glasses, headband, in-ear device), and implantable devices (e.g., subcutaneous implanted device). The case manager computing devices 20 or the individual computing devices 22, or both can, for example, each respectively store one or more unique identifiers, for example, a Mobile identification Number (MIN), Mobile Subscription Identification Number (MSIN), Mobile Station. Identifier (MSID) international Mobile Subscriber identity (IMSI), or International Mobile Station Equipment identity (IMEI). Such can be stored in the device in a non-removable memory or in a removable memory (e.g., Subscriber Identity Module (SIM) card). In some examples, case manager computing devices 20 and/or individual computing devices 22 may include a global positioning system (GPS) device, for example, GPS device 106 shown in FIG. 1. In some examples, a GPS device may be included in automobiles 14. The GPS devices may provide GPS-verified check-ins in some examples.

Each of the case manager computing devices 20, individual computing devices 22, or recovery server 28 may communicably couple to the communications network 24 via at least one respective wired connection (e.g., electrically conductive wires, optical fiber) or at least one respective wireless connection (e.g., radio, WI-FI® radio, Bluetooth® radio, cellular radio, optical emitter and sensor pair such as an infrared emitter and sensor pair). The recovery server 28 can communicably couple to at least one of case manager computing devices 20 or individual computing devices 22 via the communications network 24. The case manager computing devices 20 ands or individual computing devices 22 may provide inputs to the recovery server 28 via the communications network 24 from the variety of facilities and locations (e.g., facility 6a, park 6h, facility 6n, residence 16, automobile 14c). In some examples, case managers 10 may provide inputs to the recovery server 28 via case manager computing devices 20 for non-connected individuals.

The recovery server 28 may store inputs received from the case manager computing devices 20 and or individual computing devices 22 in a non-transitory computer-readable memory 32b. In some examples, computer-readable memory 32b may include memory 114 shown in FIG. 1. The recovery server 28 may provide outputs (e.g., risk scores, rewards), to case manager computing devices 20 and/or individual computing devices. In some examples, additional recovery servers 26 and 30 may be included in the environment 4. In some examples, the each of the recovery servers 26, 28, and 30 may only be accessible to certain individuals 12 and/or case managers 10. In some examples, the recovery servers 26, 28, and 30 may be accessible to all individuals 12 and/or case managers 10. In these examples, the recovery servers 26, 28, and 30 may communicate via the communications network 24 to maintain synchrony of data between the recovery servers. In some examples, each of the recovery servers 26, 28, and 30 may be accessible to certain individuals 12, but all of the recovery servers 26, 28, and 30 may be accessible to all of the case managers 10.

As shown in FIG. 9, individuals 12 in recovery may attend support activities at various locations (e.g., facility 6a, 6n) and check-in via an individual computing device 22. individuals may also engage in self-care activities at various locations (e.g., park 6h, residence 16) and check-in via the individual computing device 22. Case managers 10 may view risk scores for one or more individuals 8, 12 via case manager computing device 20 from a variety of locations (e.g., facility 6a, 6n). Case managers 10 may meet with individuals 8, 12 under their care at various locations (e.g., facility 6a, 6n). In some examples, the case managers 10 may alter the individual 8, 12, the location, and/or timing of meetings based on the risk score.

The systems e.g., systems 2 and 100) and methods described herein may be used to support individuals in addiction recovery. For example, a case manager (e.g., case manager 10) may use the systems and methods described herein to calculate a risk score for one or more individuals under their care (e.g., individuals 8, 12). The risk score may be a quantitative measure of risk of relapse of an individual. A quantitative measure of risk of relapse provided by the systems and methods described herein may allow the case manager to more reliably provide a proper level of care to the individuals in their case load compared to qualitative assessments. For example, a case manager may view a risk score for an individual.

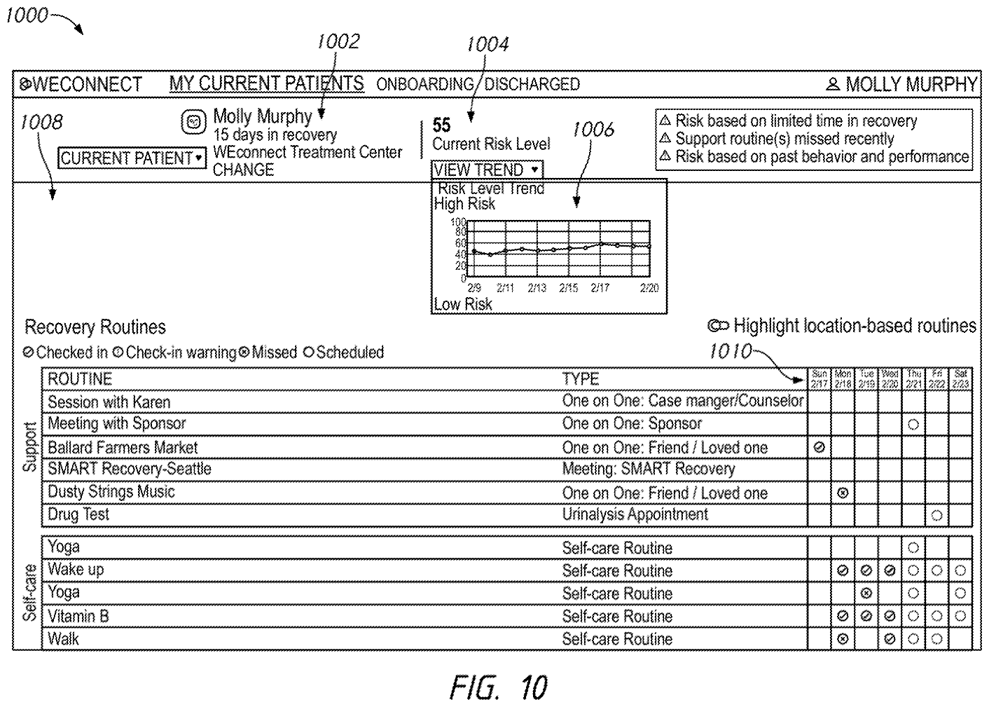

FIG. 10 shows a screenshot 1000 of an individual's information from a case manager computing device in accordance with examples described herein. In addition to viewing an individual's risk score 1004, the case manager may view other information such as the individual's personal information 1002, plot 1006 of the individual's historical risk score, scheduled support and self-care activities 1008, and/or check-in history 1010. Additional information, such as prior SOS requests and/or medical information, may be viewed in other examples.

If the individual's risk score is low, the case manager may take no action or maintain a current recovery plan (e.g., checking in with the individual by phone once a week). If the individuals risk score is high, the case manager may take further action. For example, the case manager may reach out to the individual by text or phone, prescribe or adjust a dosage of a medication, contact a medical professional, and/or schedule additional support activities. Whether a risk score is considered high or low may be based on a threshold value. In some examples, scores equal to or above 50 may be considered high and scores below 50 may be considered low. Other threshold values may be used.

In some examples, when the risk score is above a threshold, the action taken by the case manager may vary depending on how high above the threshold the risk score is. For example, if the threshold value is 50, and an individuals risk score is 60, the case manager may contact the individual by e-mail and/or schedule an additional support activity. If the individuals risk score is 95, the case manager may physically meet with the individual and/or dispatch medical professionals to meet with the individual.

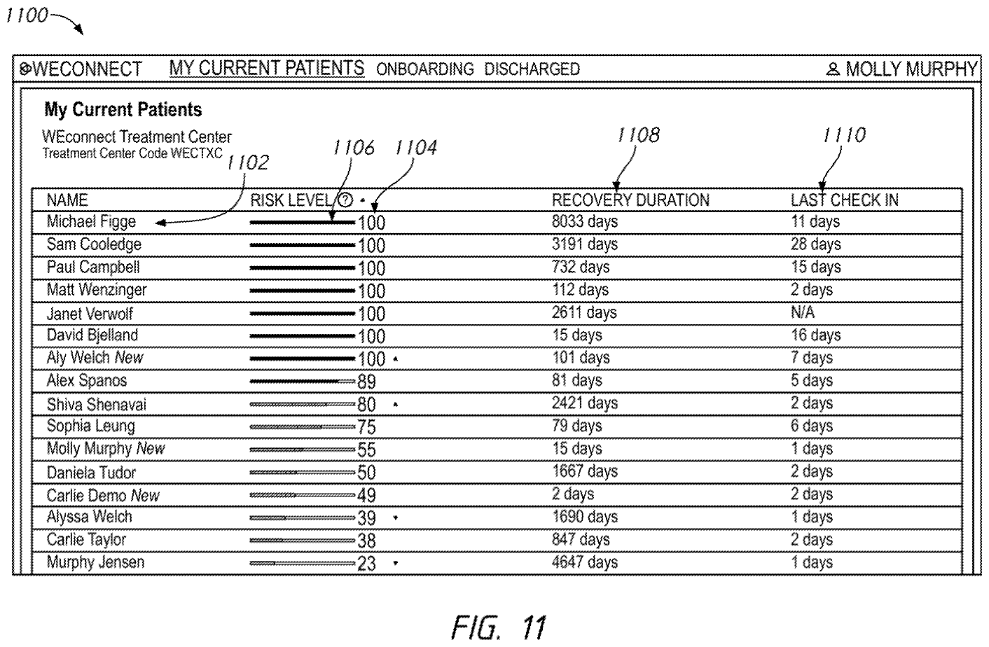

In another example, a case manager may view risk scores for multiple individuals, FIG. 11 is a screenshot 1100 of multiple individuals' information from a case manager computing device in accordance with examples described herein. The case manager may view a list of individuals 1102 and risk scores 1104. In the example shown in FIG. 11, the risk score 1104 is accompanied by a visual representation 1106 of the risk score. In this example, the visual representation 1106 is a bar that increases in length as the risk score increases. The bar may also change color as the risk score increases (e.g., green for low scores, orange for intermediate scores, and red for high scores). Other types of visual representations may be used in other examples (e.g., pie charts, happy/sad faces). In other examples, the visual representation 1106 may be omitted, in some examples, the case manager may view additional information. In the example shown in FIG. 11, the case manager may view the number of days in recovery 1108 for each individual and the last time the individual checked in 1110. Other information, such as number of activities and/or check-in rates, may be viewed in other examples

Based on the ranking of individuals by their risk scores, the case manager may take actions directed to individuals with higher risk scores prior to taking actions directed to individuals with lower risk scores. For example, the case manager may prioritize individuals with higher risk scores when scheduling, appointments with medical professionals e.g., individuals with higher risk scores may have earlier appointments). In another example, the case manager may budget more time for appointments with individuals with higher risk scores (e.g., an hour) compared to appointments with individuals with lower risk scores (e.g., thirty minutes). In a further example, based on the individuals' risk scores, the case manager may suggest that individuals with high risk scores temporarily disassociate from other individuals with high risk scores.

In some examples, if a case manager finds a significant number of the individuals under their care have high risk scores, the case manager may re-assign some of the individuals under their care to other case managers to ensure all of the individuals in recovery receive adequate care from case managers. In some examples, risk scores of individuals may be used for initial assignment of individuals to case managers to balance the number of high risk and low risk individuals under each case manager's care.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the disclosure. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing horn the broader and intended spirit and scope of the present system as set forth in the claims that follow. Accordingly, the specification and drawings are to be regarded in an illustrative manner and are not intended to limit the scope of the appended claims.

What is claimed is:

1. A method of determining an individual in addiction recovery's risk of relapse, the method comprising:
receiving a plurality of inputs including GPS-verified activity check-ins for the individual;
calculating a plurality of risk factors, based at least in part, on the plurality of inputs, wherein a numerical value of the plurality of risk factors is further based at least in part on a number of days the individual has been in recovery;
assigning weights to individual ones of the plurality of risk factors;
multiplying the plurality of risk factors by the corresponding weights assigned to generate a corresponding plurality of weighted factor values; and
summing the weighted factor values to generate a risk score, wherein the risk score indicates to a case manager a quantitative assessment of the individual's risk of relapse.

2. The method of claim 1, wherein the plurality of inputs include: the number of days in recovery, a volume of support activities, a volume of self-care activities, a historical risk score a support activity check-in rate, and a self-care activity check-in rate, wherein the support activity check-in rate and the self-care activity check-in rate are based, at least in part, on the GPS-verified activity check-ins.

3. The method of claim 2, wherein the plurality of inputs further includes a number of days since the individual made an SOS request.

4. The method of claim 2, wherein a risk factor associated with the number of days in recovery is assigned a highest weight of the weights.

5. The method of claim 2 wherein a risk factor associated with the historical risk score and a risk factor associated with the number of days in recovery are assigned equal weights.

6. The method of claim 2, wherein a weight assigned to a risk factor associated with the support activity check-in rate is greater than a weight assigned to a risk factor associated with the self-care activity check-in rate.

7. The method of claim 2, wherein a risk factor associated with the number of days in recovery decreases as the number of days in recovery increases.

8. The method of claim 7, wherein the risk factor associated with the number of days in recovery decreases exponentially.

9. The method of claim 2, wherein a first risk factor associated with the support activity volume is based on a number of support activities per week and the number of days in recovery and a second risk factor associated with the self-care activity volume is based on a number of self-care activities per week and the number of days in recovery.

10. The method of claim 9, wherein the number of support activities per week required to generate a low value for the first risk factor decreases as the number of days in recovery increases, and wherein the number of self-care activities per week required to generate a low value for the second risk factor decreases as the number of days in recovery increases.

11. The method of claim 2, wherein a first risk factor associated with the support activity check-in rate increases as the support activity check-in rate decreases and wherein a second risk factor associated with the self-care activity check-in rate increases as the self-care activity check-in rate decreases.

12. The method of claim 2, wherein a risk factor associated with the historical risk score is proportional to the historical risk score.

13. The method of claim 1, further comprising:
assigning sensitivity values to individual ones of the plurality of risk factors, wherein the sensitivity values are proportional to target proportions that individual ones of the plurality of risk factors contribute to the risk score;
scaling the plurality of risk factors by the sensitivity values to generate a plurality of sensitized factor values;
calculating differences between each of the plurality of sensitized factor values and the risk score and summing the differences to generate a delta sum; and
adding the delta stun to the risk score to generate an adjusted risk score.

14. A system for determining an individual in addiction recovery's risk of relapse, the system comprising:
a non-transitory computer-readable medium configured to store a plurality of inputs including GPS-verified activity check-ins for the individual;
a processor communicatively coupled to the non-transitory computer-readable medium, the processor configured to:
calculate a plurality of risk factors, based at least in pan, on the plurality of inputs, wherein a numerical value of the plurality of risk factors is further based at least in part on a number of days the individual has been in recovery;
assign weights to individual ones of the plurality of risk factors;
multiply the plurality of risk factors by the corresponding weights assigned to generate a corresponding plurality of weighted factor values; and
sum the weighted factor values to generate a risk score, wherein the risk score quantifies the individual's risk of relapse; and
a display configured to provide the risk score to a case manager.

15. The system of claim 14, wherein at least one of the plurality of inputs is provided by the individual.

16. The system of claim 14, wherein the display further provides values indicating how individual ones of the plurality of inputs contributed to the risk score.

17. The system of claim 14, wherein the risk score is further provided to the individual.

18. The system of claim 14, wherein the plurality of inputs include: the number of days in recovery, a volume of support activities, a volume of self-care activities, a historical risk score a support activity check-in rate, and a self-care activity check-in rate, wherein the support activity check-in rate and the self-care activity check-in rate are based, at least in part, on the GPS-verified activity check-ins.

19. The system of claim 18, wherein risk factors associated with the support activity check-in rate, the self-care activity check-in rate, the volume of support activities, and the volume of self-care activities are based on inputs from the plurality of inputs received within a week of generating the risk score.

20. The system of claim 14, wherein the processor is further configured to:
assign sensitivity values to individual ones of the plurality of risk factors, wherein the sensitivity values are proportional to target proportions that individual ones of the plurality of risk factors contribute to the risk score;
scale the plurality of risk factors by the sensitivity values to, generate a plurality of sensitized factor values;
calculate differences, between each of the plurality of sensitized factor values and the risk score and summing the differences to generate a delta sum; and
add the delta sum to the risk score to generate an adjusted risk score,
wherein the display is further configured to provide the adjusted risk score to the case manager.

21. The system of claim 20, further comprising an input device configured to receive inputs from the case manager to adjust at least one of the weights or sensitivity values.

22. A method of triaging individuals in addiction recovery based on a risk of relapse, the method comprising:
receiving a plurality of inputs including GPS-verified activity check-ins for the individuals;
calculating a plurality of risk factors for each individual, based at least in part, on the plurality of inputs, wherein a numerical value of the plurality of risk factors is further based at least in part on a number of days each individual has been in recovery;
assigning weights to individual ones of the plurality of risk factors;
multiplying the plurality of risk factors by the corresponding weights assigned to generate a corresponding plurality of weighted factor values for each individual;
summing the weighted factor values to generate a risk score for each individual; and
ranking the individuals from a highest priority to a lowest priority based on the risk score for each individual, wherein highest priority individuals are at a greater risk of relapse than the lowest priority individuals.

23. The method of claim 22, wherein the plurality of inputs include: the number of days in recovery, a volume of support activities, a volume of self-care activities, a historical risk score a support activity check-in rate, and a self-care activity check-in rate, wherein the support activity check-in rate and the self-care activity check-in rate are based, at least in part, on the GPS-verified activity check-ins.

24. The method of claim 22, further comprising:
assigning sensitivity values to the individual ones of the plurality of risk factors, wherein the sensitivity values are proportional to target proportions that individual ones of the plurality of risk factors contribute to the risk score;
scaling the plurality of risk factors by the, sensitivity values to generate a plurality of sensitized factor values for each individual;
calculating differences between each of the plurality of sensitized factor values and the risk score and summing the differences to generate a delta sum for each individual;
adding the delta sum to the risk score to generate an adjusted risk score for each individual; and
adjusting the ranking of the individuals based on the adjusted risk scores.

25. A method of providing a reward to an individual in addiction recovery, the method comprising:
providing a challenge to the individual based at least in part on a number of days the individual has been in recovery, wherein the challenge includes at least one of a volume of support activities to be completed in a period of time or a volume of self-care activities to be completed in the period of time;
receiving at least one of a support activity cheek-in rate or a self-care activity check-in rate, wherein the support activity check-in rate is based on UPS-verified check-ins for the individual;
determining, based at least in part on the at least one of the support activity check-in rate or the self-care activity check-in rate, whether the challenge was completed; and
if it is determined the challenge was completed, providing the reward to the individual.

26. The method of claim 25, wherein the self-care activity check-in rate is based, at least in part, on the GPS-verified check-ins for the individual.

27. The method of claim 25, wherein the reward is a monetary reward.

28. The method of claim 27, wherein the monetary reward is provided digitally to an individual computing device.
